(19) United States
(12) Patent Application Publication
(10) Pub. No.: US 2020/0383560 A1
(43) Pub. Date: Dec. 10, 2020

(54) INTRAORAL SURGICAL MOUTH PROP

(71) Applicant: Robinson Healthcare Limited, Worksop (GB)

(72) Inventor: Ralph Paul Day, Worksop (GB)

(21) Appl. No.: 16/436,029

(22) Filed: Jun. 10, 2019

(51) Int. Cl. A61B 1/24

(52) U.S. Cl. CPC A61B 1/24

(57) ABSTRACT

An intraoral surgical prop is manufactured entirely from two different Shore A hardness variants of the same high performance biocompatible sterilisable thermoplastic elastomer. It comprises an outer anatomically shaped compressible surgical prop body component (1) and an inner integrally-moulded, permanently attached, flexible and extensible tether and tether grip component (2, 3) with a thumb grip indentation (4) moulded such that it retains a permanently curved profile.

### FIELD OF THE INVENTION

This invention relates to intraoral surgical mouth props and has for its object the provision of an intraoral surgical mouth prop having enhanced features which significantly improve functionality, intraoperative performance and patient safety.

### BACKGROUND OF THE INVENTION

Maintaining clinical access to the oropharyngeal cavity during general or local anaesthesia is traditionally achieved through the insertion of a mouth prop or spacer between the upper and lower dentitions to maintain sufficient space for the procedure to be completed safely and effectively for both patient and operator.

Traditional intraoral surgical props/spacers are typically manufactured to a design specification originally described in 1940. For example, U.S. Pat. No 2,220,674 discloses a mouth prop comprising a rigid non-compressible metal substructure formed from a sheet metal blank and a moulded polymeric cover, typically a natural or synthetic rubber polymer.

A “String” or “Cord” is temporarily attached to the main body of the mouth prop as a means of assisting its removal from the oral cavity as the jaws are attempting to return to the normal, closed position post-operatively.

Such traditional designs are associated with significant clinical shortcomings, primarily relating to the unsafe and inefficient removal of intraoral surgical mouth props. Removal is typically achieved by the operating clinician needing to pull upon either a cord or steel chain temporarily attached to the main body of the mouth prop by means of a compression-fit steel ring, in the case of a chain, or knotted loop, in the case of a cord. This manoeuvre often requires significant force, and as a result of the repeated stresses exerted upon the cord or chain during removal causes progressive tensile strength degradation, which in turn often results in inadvertent breakage or detachment of the cord or chain, leaving the mouth prop body in situ, thereby creating major clinical difficulties with ensuring its safe and effective removal.

Reusable mouth prop chains are also typically manufactured from sub-optimal grades of steel which are prone to rusting as a direct result of being subjected to the repeated washing and steam sterilisation cycles required between clinical procedures, thereby creating problems with managing patient safety, optimal infection prevention, and costly maintenance.

Intraoral surgical mouth props of traditional design are also associated with additional design shortcomings, which include lack of visibility in the operative field, inability to easily identify and change specific mouth prop sizes intraoperatively and causation of trauma to intraoral soft and hard tissues as a direct result of inappropriately contoured product geometry (which is traditionally unsympathetic to the fragile nature of adjacent anatomical structures).

U.S. Pat. No. 5,466,153 discloses a bite block mouth prop featuring anatomically contoured outer elements and an inner element extending from an anterior surface to an integral tether in the form of a handle which is bent to form a grip. Patent Specification No. WO 9718002 discloses a bite block featuring an anatomically contoured surface formed of a medical grade plastic and an interior element extending to a tether in the form of a handle with a grip.

It is an object of the present invention to provide an intraoral surgical mouth prop the design of which is such as to significantly enhance the functionality, intra operative performance and safety of such products, thereby providing a significantly optimised and fit for purpose medical device.

### SUMMARY OF THE INVENTION

According to a first aspect of the present invention there is provided an intraoral surgical mouth prop comprising:

an anatomically contoured compressible outer mouth prop body element, which includes bite grip formations and flanges designed to keep the cheeks and tongue away from the bite grip formations, and

an inner body element that is less compressible than the outer body element and includes a portion that fits within the outer body element and a flexible tether that is of curved form and has a grip at the end thereof remote from said portion.

According to a second aspect of the present invention there is provided an intraoral surgical mouth prop comprising:

an anatomically contoured compressible outer mouth prop body element, which includes bite grip formations and flanges designed to keep the soft tissues of the cheeks and tongue away from the bite grip formations, and

an integral inner element comprising a permanently attached less compressible and extensible flexible tether and tether grip, the outer element having a Shore A hardness within the range Shore A 55 to Shore A 75 and the inner element having a Shore A hardness with the range Shore A 85 to Shore A 95.

The intraoral surgical mouth prop is preferably manufactured entirely from two different Shore A hardness variants of the same high performance biocompatible sterilisable thermoplastic elastomer using an over-moulding or twin-shot co-injection moulding technique. The mouth prop may be a single use mouth prop designed to facilitate the safe and effective insertion and removal of the mouth prop, which entirely eliminates the risks of inadvertent chain or cord breakage or detachment and cross infection, typically associated with traditional reusable intraoral surgical mouth props.

The permanently attached integral flexible and extensible tether/tether grip element is preferably moulded in such a way that it follows a permanently curved form, to minimise any potential interference with operative field access.

### BRIEF DESCRIPTION OF THE DRAWINGS

FIG. 1 is a side elevation of the device describing its principal inventive step, an over-moulded or twin shot co-injection moulded integral permanently attached flexible and extensible tether and tether grip,

FIG. 2 is a side elevation of the device detailing the curved profile of the moulded integral tether at its points of insertion with the mouth prop body and the tether grip,

FIG. 3 is a plan view detailing the profile of the moulded integral tether and tether grip and moulded bite grips on the occluding surface of the mouth prop,

FIG. 4 is a side elevation of the mouth prop body detailing the moulded aperture which provides a means of passage and location for intraoral aspiration devices of multiple dimension and design,

FIG. 5 is a magnified plan view of the mouth prop body detailing the moulded bite grips on the occluding surface of the mouth prop,

FIG. 6 is a side elevation with cross hatched area describing the rising internal to external taper of the bite surface profile, and

FIGS. 7, 8 and 9 are perspective views of the three physiologically-dimensioned intraoral surgical mouth prop variants.

### DESCRIPTION OF THE PREFERRED EMBODIMENTS

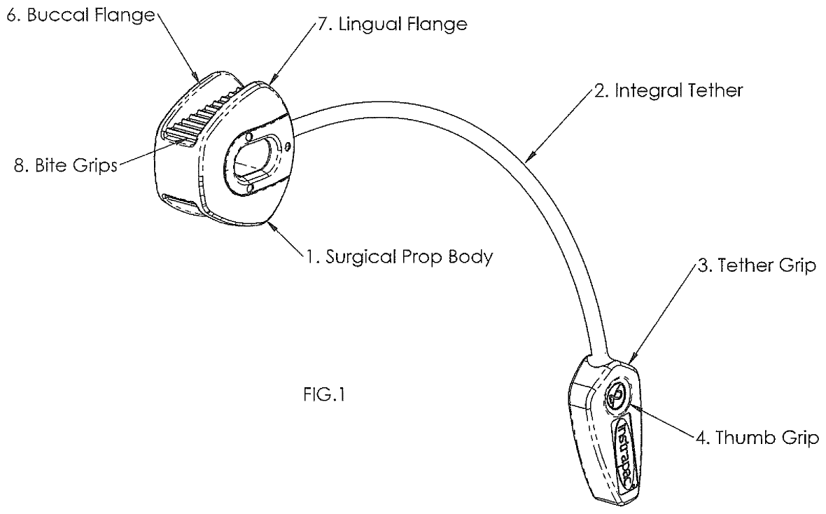

FIG. 1 of the drawings shows a single use intraoral surgical prop, comprising an outer anatomically shaped compressible surgical prop outer body element 1 and a less compressible inner integrally-moulded inner body element fits within the outer body element and a permanently attached, flexible and extensible tether 2 and tether grip 3 with thumb grip indentation 4, designed to facilitate the safe and effective insertion and removal of the single use intraoral surgical prop, and to eliminate the risk of breakage or detachment and cross-infection associated with traditional reusable cords and chains.

FIG. 1 of the drawings shows the novel inner mouth prop body element comprising an integral tether 2, tether grip 3 and thumb grip formation 4 and the outer mouth prop body element comprising the surgical prop body 1, within which a portion of the inner body element is contained, and two protective flexible anatomically contoured flanges 6, 7 of novel profile located on the lateral aspects of the bite grip platforms 8. These flanges 6, 7 are designed to hold the soft tissues of the cheek and tongue away from the bite grip platforms 8 when the teeth are in occlusion, thereby preventing iatrogenic soft tissue trauma.

The inner and outer body elements of the intraoral surgical mouth prop are manufactured from two different Shore A hardness variants of the same thermoplastic elastomer. The outer element 1, 6, 7 and 8 is more compressible (for example, within the range Shore A 55 to Shore A 75) and the inner element 2, 3 and 4 is less compressible and extensible (for example, within the range Shore A 85 to Shore A 95). Such differential compression imparts the necessary physical properties required of an optimally designed intraoral surgical mouth prop, whereby the outer body element is sufficiently compressible to ensure that the teeth are held securely in occlusion without causing damage to the dentition, and the inner element is less compressible, providing the necessary core strength to resist the significant compressive forces exerted upon the mouth prop body when the teeth are in full occlusion.

The intraoral surgical mouth prop is preferably manufactured from two Shore A hardness grades of the same high performance biocompatible sterilisable thermoplastic elastomer such as, but not restricted to, TPU, i.e. thermoplastic urethane, TPS (TPE-s), TPO (TPE-o), TPV (TPE-v or TPV), TPC (TPE-E), TPA (TPE-A) , TPZ or alternatively silicone rubber, using an over-moulding or twin-shot co-injection moulding technique.

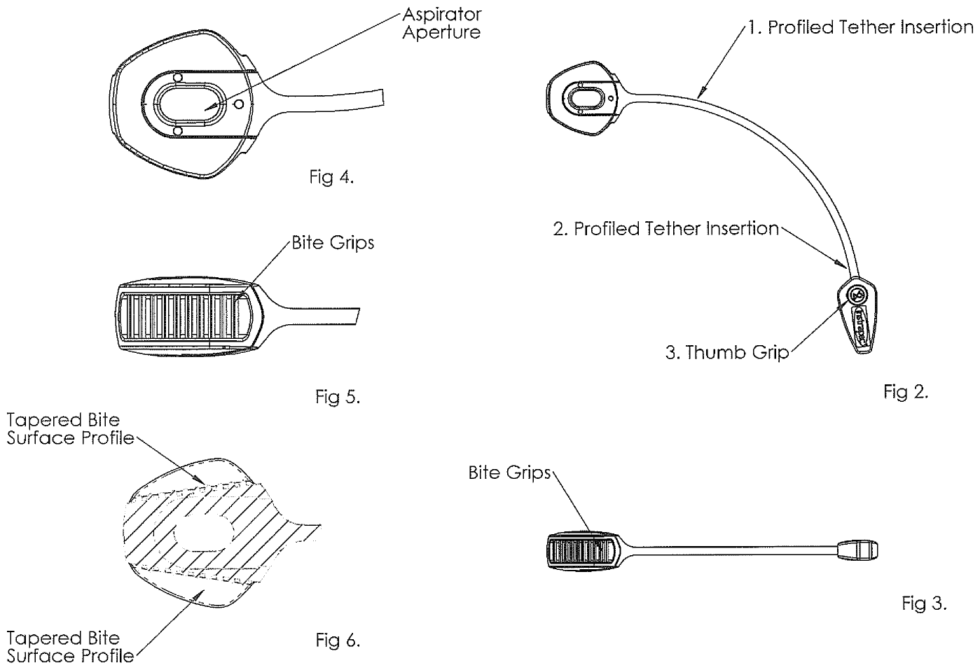

FIG. 2 of the drawings shows the moulding profile of the flexible and extensible tether at the points where it meets the mouth prop body 1 and the tether grip 2 to provide a smooth atraumatic transition profile, thereby minimising potential damage to fragile intraoral soft tissues when in situ.

FIG. 2 of the drawings also shows that the integral tether and tether grip is moulded in such a way that it follows a permanently curved form so that, when in situ, it lies in an inferior plane to the lower lip of the person in whose mouth the prop has been inserted, to minimise any potential interference with access to the operative field.

FIGS. 3 and 5 of the drawings show stepped bite grip platforms 8 located on the upper and lower surfaces of the mouth prop body, providing for atraumatic locating surfaces for the upper and lower teeth to prevent unwanted movement of the prop and damage to the teeth when in situ.

FIG. 4 of the drawings shows a central “aspirator aperture” located within the inner body element. This aperture is designed to impart two specific properties to the device. In the first instance, it provides the prop with the ability to selectively compress when the upper and lower teeth are located in occlusion, thereby reducing potential trauma to the dentition. In the second instance, the geometry and dimensions of said aperture provide a means of passage and location for intraoral aspiration devices of multiple dimension and design to facilitate the efficient removal of saliva, blood, tissue fluids and surgical hand piece irrigation fluids from the operative field, thereby facilitating enhanced intra-operative visibility for the clinician and reduced compromise of the patient's airway, particularly when the operating clinician is operating without assistance.

FIG. 6 of the drawings shows the rising internal to external taper of the bite grip platforms 8, which ensures that the upper and lower dentitions may be separated in a safe and atraumatic open position during insertion and final placement of the mouth prop.

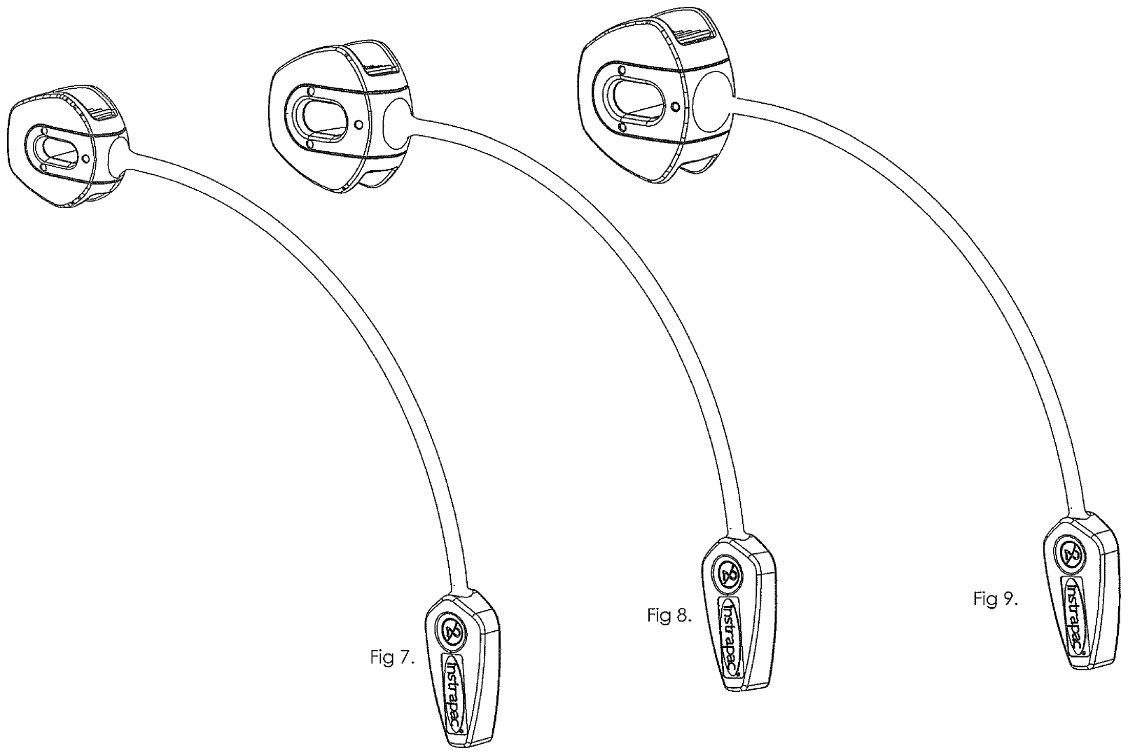

FIGS. 7, 8 and 9 of the drawings show perspective views of three discrete sizes of the intraoral surgical mouth prop which are designed to replicate the average intraoral anatomical and physiological dimensions of a child, medium-sized adult or large-sized adult. These drawings also show that the dimensions and profile of the integral tether and tether grip are specifically designed to be common across all three sizes, thereby ensuring that the tether 2 possesses the required elongation to break and tensile strength properties to facilitate the safe and effective removal of the surgical prop from the oral cavity, including sudden removal in an emergency situation, without detachment or breakage.

Three size variants of the intraoral surgical mouth prop may be produced, such that the outer prop body component is moulded in a preferably white thermoplastic elastomer to provide enhanced visibility in the operative field, and the inner component comprising the integral tether/tether grip/aperture core is moulded in the same thermoplastic elastomer in a contrasting colour to facilitate rapid intra-operative visual identification of each size (For example, Large Adult size: White Outer element, Blue inner element, Medium Adult size: White Outer element, Green inner element, Child size: White Outer element, Purple inner element).

1. An intraoral surgical mouth prop comprising:
an anatomically contoured compressible outer mouth prop body element, which includes bite grip formations and flanges designed to keep the cheeks and tongue away from the bite grip formations, and
an inner body element that is less compressible than the outer body element and includes a portion that fits within the outer body element and a flexible tether that is of curved form and has a grip at the end thereof remote from said portion.

2. A mouth prop as claimed in claim 1, manufactured entirely from two different Shore A hardness variants of the same high performance biocompatible thermoplastic elastomer.

3. A mouth prop as claimed in claim 2, produced using an over-moulding or twin-shot co-injection moulding technique.

4. A mouth prop as claimed in claim 1, in which the portion of the inner body element that is fits within the outer body element includes an aspirator aperture.

5. An intraoral surgical mouth prop comprising:
an anatomically contoured compressible outer mouth prop body element, which includes bite grip formations and flanges designed to keep the soft tissues of the cheeks and tongue away from the bite grip formations, and
an integral inner element comprising a permanently attached less compressible and extensible flexible tether and tether grip, the outer element having a Shore A hardness within the range Shore A 55 to Shore A 75 and the inner element having a Shore A hardness with the range Shore A 85 to Shore A 95.
